(19) United States
(12) Patent Application Publication
(10) Pub. No.: US 2023/0160869 A1
(43) Pub. Date: May 25, 2023

(54) SYSTEM FOR MONITORING ELECTRICAL PROPERTIES OF MATERIALS OF DEEP BEDROCK SAMPLES FOR ESTIMATING NUCLIDE MOVEMENT IN DISPOSAL SITE OF SPENT FUEL

(71) Applicant: KOREA RADIOACTIVE WASTE AGENCY, Gyeongju-si (KR)

(72) Inventors: Hae Ryong JUNG, Daejeon (KR); Min Seok KIM, Daejeon (KR); Chang Min SHIN, Daejeon (KR); Seung Hyun KIM, Daejeon (KR); Hyung Ju YUN, Daejeon (KR); Sang Hwan LEE, Daejeon (KR); Man Ho HAN, Daejeon (KR); Jung Hwan LEE, Daejeon (KR)

(21) Appl. No.: 17/993,965

(22) Filed: Nov. 24, 2022

(30) Foreign Application Priority Data
Nov. 25, 2021 (KR) 10-2021-0164523

(51) Int. Cl. G01N 33/24; G01N 27/04; G01T 1/167

(52) U.S. Cl. CPC G01N 33/24; G01N 27/041; G01T 1/167

(57) ABSTRACT

The present invention relates to a system for monitoring electrical properties of materials of deep bedrock samples for estimating nuclide movement in disposal site of spent fuel and, more specifically, to a system for monitoring electrical properties of materials of deep bedrock samples for estimating nuclide movement in disposal site of spent fuel which installs a tube wherein ring-shaped potential electrode is formed in multi layers inside a column; adheres the potential electrode to deep bedrock samples by pressing on the external side of the tube while filling the deep bedrock samples inside of the tube; and reproduces real condition of deep bedrock and monitors precisely by measuring electrical resistivity for each location of the deep bedrock samples while injecting nuclide and underground water to the inside the tube.

### TECHNICAL FIELD

The present invention relates to a system for monitoring electrical properties of materials of deep bedrock samples for estimating nuclide movement in disposal site of spent fuel and, more specifically, to a system for monitoring electrical properties of materials of deep bedrock samples for estimating nuclide movement in disposal site of spent fuel which installs a tube wherein ring-shaped potential electrode is formed in multi layers inside a column; adheres the potential electrode to deep bedrock samples by pressing on the external side of the tube while filling the deep bedrock samples inside of the tube; and reproduces real condition of deep bedrock and performs precise monitoring by measuring electrical resistivity for each location of the deep bedrock samples while injecting nuclide and underground water to the inside the tube.

### BACKGROUND ART

Referring to waste which comes from nuclear facilities and workplaces or laboratories, etc., handling radioactive materials, radioactive waste is classified into low and intermediate level radioactive waste and high level radioactive waste depending on degrees polluted by radioactive materials. Relevant Act stipulates that the definition of radioactive waste refers to radioactive materials or materials polluted by radioactive materials, resulting in materials to be eliminated. Examples are work items, etc., polluted during maintenance in nuclear facilities, items polluted during treatment in hospitals, and non-destructive inspections, etc., in industries. Further, the high-level radioactive waste is nuclear fuel, etc., which remains from power production in nuclear power plants. That is, radioactive waste is generated from all types of fields in which nuclear power is used in schools, hospitals, research institutes, industries, nuclear power plants, etc.

Disposal method of the low and intermediate level radioactive waste considers characteristics of underground hundreds of meters below depending on cases. Near Surface Disposal refers to a method of installing and reclaiming concrete vaults on the surface of the ground, and Rock Cavern Disposal is a method of excavating tunnels and silos to underground rock at least more than tens to hundreds of meters and reclaiming the inside for disposal. As for high-level radioactive waste (spent nuclear fuel), deep geological disposal is performed in the bedrock at a depth of 500˜1,000 meters.

As for high-level radioactive waste (spent nuclear fuel), estimation on disposal site should be preceded.

Explaining on the near surface disposal more specifically, new disposal facilities for decommissioning radioactive waste disposal of large-scale nuclear power plants have been planning and under construction in Gyeongju radioactive waste disposal. Among them, the near surface disposal, so called second-stage disposal, is targeted at below low level waste, currently promoting licensing.

In the second-stage near surface facility, a concrete disposal vault is constructed on the ground and a disposal container having radioactive waste is stored inside the disposal vault. Also, after finally finishing disposal, the vault is placed underground in a way of a technological wall which consists of soil, etc.

Further, upon selecting disposal site of spent nuclear fuel, experimental apparatus and methods for making effective estimation prior to field application is now required.

Meanwhile, prior arts for measuring electrical resistivity toward general bedrock samples have been disclosed.

For example, Korean Patent Registration No. 10-0926318 relates to a holder for measuring electrical resistivity and apparatus for measuring electrical resistivity; Korean Patent Registration No. 10-1698036 relates to a holder for core sample, resistance measurement apparatus and seismic velocity measurement apparatus for the core sample using the same; Korean Patent Registration No. 10-1003755 relates to a method for analysis of pollutant transport in soil, and etc.

The holder for measuring electrical resistivity and apparatus for measuring electrical resistivity relates to the holder for measuring electrical resistivity and apparatus for measuring electrical resistivity for measuring electrical resistivity of rock samples, and more particularly, compresses both ends of rock samples with a constant pressure and measures electrical resistivity of rock samples while keeping moisture content of rock samples more than a constant degree.

The holder for core sample, resistance measurement apparatus and seismic velocity measurement apparatus for the core sample using the same measures a support for fixing extremely precise location of the core sample, resistance and seismic velocity using the same.

The method for analysis of pollutant transport in soil enables to rapidly recognize results without chemical analysis of outflow water and reproduce pollutant transport in the soil and underground water more directly and more realistic, by suggesting testing apparatus for analysis of pollutant transport in soil.

However, the holder for measuring electrical resistivity and apparatus for measuring electrical have a problem with exclusion of electrode polarization effects in that current electrode and potential electrode use the identical electrode; the holder for core sample, resistance measurement apparatus and seismic velocity measurement apparatus for the core sample using the same have a problem with a large margin in case of a medium which has the lowest resistance even though the resistance in the holder is measured by a two electrode method; and in the method for analysis of pollutant transport in soil, it is hard to represent the whole inside the column based on fluid transport in soil merely through an extremely small monitoring electrode, installed.

### PRIOR ART

(Patent document 001) Korean Patent Registration No. 10-0926318
(Patent document 002) Korean Patent Registration No. 10-1698036
(Patent document 003) Korean Patent Registration No. 10-1003755

### DISCLOSURE

### Technical Problem

For solving above problems, the object of the present invention is to provide a system for monitoring electrical properties of materials of deep bedrock samples for estimating nuclide movement in disposal site of spent fuel which installs a tube wherein ring-shaped potential electrode is formed in multi layers inside a column; adheres the potential electrode to deep bedrock samples by pressing on the external side of the tube while filling the deep bedrock samples inside of the tube; and reproduces real condition of deep bedrock and performs precise monitoring by measuring electrical resistivity for each location of the deep bedrock samples while injecting nuclide and underground water to the inside the tube.

Further, the another object of the present invention is to provide a system for monitoring electrical properties of materials of deep bedrock samples for estimating nuclide movement in disposal site of spent fuel which installs current electrode with vortex formation for having elasticity on the top and the bottom of deep bedrock samples and supplies current to a large area of the top and the bottom sides of the deep bedrock samples, thereby monitoring precisely by excluding electrode polarization effects.

Further, the another object of the present invention is to provide a system for monitoring electrical properties of materials of deep bedrock samples for estimating nuclide movement in disposal site of spent fuel which reproduces real condition of the deep bedrock samples by installing a chamber wrapping the column and controlling the temperature of the inside the chamber.

### Technical Solution

To accomplish above objects, the present invention comprises: a column for being inserted into deep bedrock samples in disposal site of spent fuel; flowing in nuclides and underground water through an upper inlet; discharging nuclides and underground water through a lower outlet; and being equipped with current electrode on the top and the bottom; a tube for covering the deep bedrock samples and installing potential electrode in multi layers inside; a metering pump for supplying underground water including nuclide to the inside of the column with a fixed amount at a constant speed; an electrical resistivity measuring instrument for supplying current to the current electrode and measuring electrical resistivity for each location of the deep bedrock samples by the potential electrode; and a main controller for controlling injection speed of the metering pump, pressure of the compressor and supply current of the electrical resistivity measuring instrument.

Hereinafter, the system for monitoring electrical properties of materials of deep bedrock samples for estimating nuclide movement in disposal site of spent fuel further comprises a web server which receives and monitors measured values, measured in the electrical resistivity measuring instrument, through wire-wireless network; and transfers control data by the main controller through wire-wireless network.

Hereinafter, the system for monitoring electrical properties of materials of deep bedrock samples for estimating nuclide movement in disposal site of spent fuel further comprises a chamber for wrapping the column; a conditioning equipment for controlling the temperature of the chamber; and a storage container for storing underground water including nuclide which is discharged from the column.

Hereinafter, the system for monitoring electrical properties of materials of deep bedrock samples for estimating nuclide movement in disposal site of spent fuel further comprises a storage container for storing underground water including nuclide which is discharged from the column.

Hereinafter, the main controller controls the temperature of the inside of the chamber by controlling the temperature of the conditioning equipment.

Hereinafter, the current electrode has vortex formation, but being in the shape of a cone for having elasticity.

Hereinafter, the potential electrode is formed in a partially sectioned C-shaped ring to control a diameter in response to contraction of the tube.

### Advantageous Effects

According to a system for monitoring electrical properties of materials of deep bedrock samples for estimating nuclide movement in disposal site of spent fuel, constituted as above, enables to install a tube wherein ring-shaped potential electrode is formed in multi layers inside a column; adhere the potential electrode to deep bedrock samples by pressing on the external side of the tube while filling the deep bedrock samples inside of the tube; and reproduce real condition of deep bedrock and monitors precisely by measuring electrical resistivity for each location of the deep bedrock samples while injecting nuclide and underground water to the inside the tube.

Further, according to the present invention, it enables to install current electrode with vortex formation for having elasticity on the top and the bottom of deep bedrock samples and supply current to a large area of the top and the bottom sides of the deep bedrock samples, thereby monitoring precisely by excluding electrode polarization effects.

Further, according to the present invention, it enables to reproduce real condition of the deep bedrock samples by installing a chamber wrapping the column and controlling the temperature of the inside the chamber.

### DESCRIPTION OF DRAWINGS

FIG. 1 illustrates an outline showing the constitution of a system for monitoring electrical properties of materials of deep bedrock samples for estimating nuclide movement in disposal site of spent fuel according to the present invention.

FIG. 2 illustrates a perspective view showing the constitution of a column in a system for monitoring electrical properties of materials of deep bedrock samples for estimating nuclide movement in disposal site of spent fuel according to the present invention.

FIG. 3 illustrates a perspective view showing the constitution of current electrode in a system for monitoring electrical properties of materials of deep bedrock samples for estimating nuclide movement in disposal site of spent fuel according to the present invention.

### DESCRIPTION OF THE PREFERRED EMBODIMENTS

The configuration of a system for monitoring electrical properties of materials of deep bedrock samples for estimating nuclide movement in disposal site of spent fuel of the present invention will be described in detail with the accompanying drawings.

In the following description of the present invention, a detailed description of known incorporated functions and configurations will be omitted when to include them would make the subject matter of the present invention rather unclear. Also, the terms used in the following description are defined taking into consideration the functions provided in the present invention. The definitions of these terms should be determined based on the whole content of this specification, because they may be changed in accordance with the option of a user or operator or a usual practice.

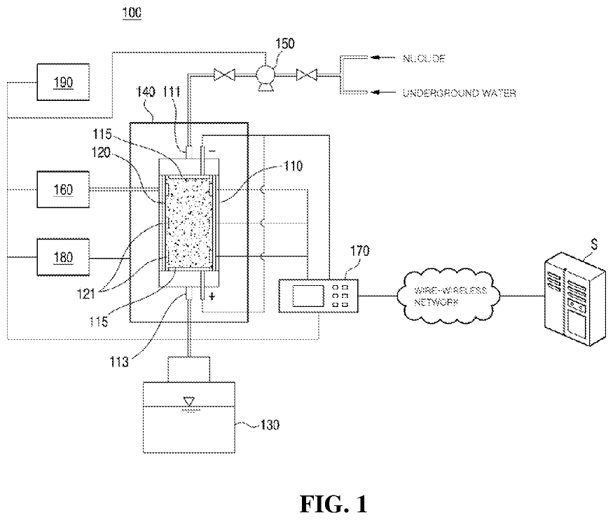

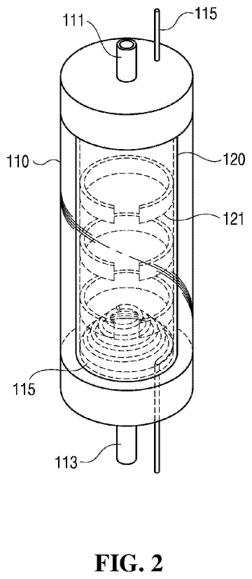

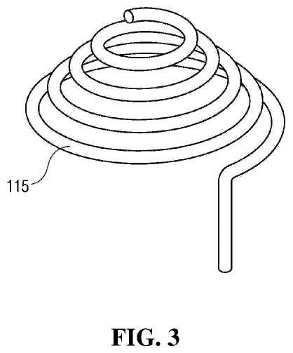

FIG. 1 illustrates an outline showing the constitution of a system for monitoring electrical properties of materials of deep bedrock samples for estimating nuclide movement in disposal site of spent fuel according to the present invention; FIG. 2 illustrates a perspective view showing the constitution of a column in a system for monitoring electrical properties of materials of deep bedrock samples for estimating nuclide movement in disposal site of spent fuel according to the present invention; and FIG. 3 illustrates a perspective view showing the constitution of current electrode in a system for monitoring electrical properties of materials of deep bedrock samples for estimating nuclide movement in disposal site of spent fuel according to the present invention.

Referring to FIGS. 1 to 3, a system for monitoring electrical properties of materials of deep bedrock samples for estimating nuclide movement in disposal site of spent fuel (100) according to the present invention consists of a column (110), a tube (120), a storage container (130), a chamber (140), a metering pump (150), a compressor (160), an electrical resistivity measuring instrument (170), a conditioning equipment (180) and a main controller (190).

First, the column (110), made of transparent materials such glass or synthetic resin, or metal, is formed in a cylindrical shape, thereby being inserted into deep bedrock samples (A) in disposal site of spent fuel; flowing in nuclides and underground water through an upper inlet (111); discharging nuclides and underground water through a lower outlet (113); and being equipped with current electrode (115) on the top and the bottom.

As illustrated in FIG. 3, the current electrode (115) has vortex formation, but being in the shape of a cone for having elasticity, and it is desirable that it is made of silver or bronze which has high conductivity and economic efficiency.

Further, formed of elastic materials for shape variation, the tube (120) covers the deep bedrock samples (A) and installs potential electrode (121) in multi layers inside.

The potential electrode (121) is formed in a partially sectioned C-shaped ring to control a diameter in response to contraction of the tube (120), and it is desirable that it is made of silver or bronze which has high conductivity and economic efficiency.

Further, the storage container (130) stores underground water including nuclide which is discharged from the column (110). Hereinafter, it is desirable that the storage container (130) is made from radiation shielding materials to prevent radiation leak.

Further, the chamber (140) wraps and seals the column (110). Hereinafter, it is desirable that the chamber (140) is made from radiation shielding materials to prevent radiation leak, and it may be filled up with deep bedrock, soil, etc., in disposal site of spent fuel inside, depending on selection.

Also, the metering pump (150) supplies underground water including nuclide with a fixed amount at a constant speed inside the column (110). Hereinafter, the metering pump (150) injects underground water at a speed of 0.1˜20/min.

Also, the compressor (160) adheres the potential electrode (121) to the external side of the round stick type deep bedrock samples (A) by contracting the tube (120), simultaneously with reproduction of real condition of deep bedrock by pressing the inside of the column (110). Hereinafter, generated from the compressor (160) and applied to the column (110), the pressure is in the range of 30˜100 kgf/cm².

Further, the electrical resistivity measuring instrument (170) supplies current to the current electrode (115) in a common way and measures electrical resistivity for each location of the deep bedrock samples (A) by the potential electrode (121).

Further, the conditioning equipment (180), as cooling and heating apparatus, controls the temperature of the chamber (140) at 10˜200° C.

Continuously, the main controller (190) controls injection speed of the metering pump (150), pressure of the compressor (160) and supply current of the electrical resistivity measuring instrument (170). Hereinafter, the main controller (190) controls the temperature of the conditioning equipment (180), thereby controlling the inner temperature of the chamber (140) to set temperature.

Meanwhile, the system for monitoring electrical properties of materials of deep bedrock samples for estimating nuclide movement in disposal site of spent fuel (100) according to the present invention comprises a web server(S) which receives and monitors measured values, measured in the electrical resistivity measuring instrument (170), through wire-wireless network; and transfers control data by the main controller (190) through wire-wireless network.

Hereinafter, the operation of the system for monitoring electrical properties of materials of deep bedrock samples for estimating nuclide movement in disposal site of spent fuel (100) according to the present invention will be described in detail with the accompanying drawing.

First, after opening the column (110), the deep bedrock samples (A) are inserted to the inside of the tube (120) and then, the column (110) is closed.

Then, while connecting each pipe and wire, the main controller (190) adheres the potential electrode (121) to the external side of the round stick type deep bedrock samples (A) by contracting the tube (120), simultaneously with reproduction of real condition of deep bedrock by controlling the compressor (160) and pressing the inside of the column (110).

In such a condition, the main controller (190) controls the metering pump (150), thereby injecting the underground water including nuclide to the inside of the column (110) at a constant injection speed and making the underground water flow down along gaps of the deep bedrock samples (A) for keeping in the storage container (130).

At the same time, the electrical resistivity measuring instrument (170) measures and stores the electrical resistivity for each location of the deep bedrock samples (A) through the potential electrode (121).

Further, the measured values of the electrical resistivity measuring instrument (170) is transferred to the web server(S) through the wire-wireless network, thereby monitoring in real time at a remote location, and a supervisor transfers control data by the main controller (190) through wire-wireless network for changing test conditions, etc., resulting in remote control.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

### EXPLANATIONS OF NUMERAL REFERENCE

110: column 115: current electrode
120: tube 121: potential electrode
130: storage container 140: chamber
150: measuring pump 160: compressor
170: electrical resistivity measuring instrument 180: conditioning equipment
190: main controller

1. A system for monitoring electrical properties of materials of deep bedrock samples for estimating nuclide movement in disposal site of spent fuel, comprising: a column for being inserted into deep bedrock samples in disposal site of spent fuel; flowing in nuclides and underground water through an upper inlet; discharging nuclides and underground water through a lower outlet; and being equipped with current electrode on the top and the bottom;
a tube for covering the deep bedrock samples and installing potential electrode in multi layers inside;
a metering pump for supplying underground water including nuclide to the inside of the column with a fixed amount at a constant speed;
an electrical resistivity measuring instrument for supplying current to the current electrode and measuring electrical resistivity for each location of the deep bedrock samples by the potential electrode; and
a main controller for controlling injection speed of the metering pump, pressure of the compressor and supply current of the electrical resistivity measuring instrument.

2. The system for monitoring electrical properties of materials of deep bedrock samples for estimating nuclide movement in disposal site of spent fuel according to claim 1, further comprising: a web server which receives and monitors measured values, measured in the electrical resistivity measuring instrument, through wire-wireless network; and transfers control data by the main controller through wire-wireless network.

3. The system for monitoring electrical properties of materials of deep bedrock samples for estimating nuclide movement in disposal site of spent fuel according to claim 1, further comprising: a chamber for wrapping the column;
a conditioning equipment for controlling the temperature of the chamber; and
a storage container for storing underground water including nuclide which is discharged from the column.

4. The system for monitoring electrical properties of materials of deep bedrock samples for estimating nuclide movement in disposal site of spent fuel according to claim 1, further comprising a storage container for storing underground water including nuclide which is discharged from the column.

5. The system for monitoring electrical properties of materials of deep bedrock samples for estimating nuclide movement in disposal site of spent fuel according to claim 3, wherein the main controller controls the temperature of the inside of the chamber by controlling the temperature of the conditioning equipment.

6. The system for monitoring electrical properties of materials of deep bedrock samples for estimating nuclide movement in disposal site of spent fuel according to claim 1, wherein the current electrode has vortex formation, but being in the shape of a cone for having elasticity.

7. The system for monitoring electrical properties of materials of deep bedrock samples for estimating nuclide movement in disposal site of spent fuel according to claim 1, wherein the potential electrode is formed in a partially sectioned C-shaped ring to control a diameter in response to contraction of the tube.
